SEQUENCE LISTING (24 sequences)

SEQ ID NO: 1 - DNA, 20 bp, Artificial Sequence
  HBV Forward Primer
gtgtctgcggcgttttatca

SEQ ID NO: 2 - DNA, 21 bp, Artificial Sequence
  HBV Reverse Primer
gacamacgggcaacatacctt

SEQ ID NO: 3 - DNA, 28 bp, Artificial Sequence
  HBV Probe
cctctkcatcckgctgctatgcctymwc

SEQ ID NO: 4 - DNA, 24 bp, Artificial Sequence
  HCV Forward Primer
aaggccttgtggtactgcctgata

SEQ ID NO: 5 - DNA, 24 bp, Artificial Sequence
  HCV Reverse Primer
cgacggttggtgttwcktttggtt

SEQ ID NO: 6 - DNA, 28 bp, Artificial Sequence
  HCV Probe
accgtgcamcatgagcacrmwtcctaaa

SEQ ID NO: 7 - DNA, 17 bp, Artificial Sequence
  Beta-actin Forward Primer
accgagcgcggctacag

SEQ ID NO: 8 - DNA, 22 bp, Artificial Sequence
  Beta-actin Reverse Primer
cttaatgtcacgcacgatttcg

SEQ ID NO: 9 - DNA, 19 bp, Artificial Sequence
  Beta-actin Probe
ttcaccaccacggccgagc

SEQ ID NO: 10 - DNA, 260 bp, Hepatitis B virus
ggacttctctcaattttctagggggatcacccgtgtgtcttggccaaaattcgcagtccccaacctccaatcactcaccaacctcctgtcctccaatttgtcctggttatcgctggatgtgtctgcggcgttttatcatattcctcttcatcctgctgctatgcctcatcttcttattggttcttctggattatcaaggtatgttgcccgtttgtcctctaattccaggatcaacaacaaccagtacgggaccatgcaaa

SEQ ID NO: 11 - DNA, 260 bp, Hepatitis B virus
ggacttctctcaattttctagggggaacacccgtgtgtcttggccaaaattcgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaaccaccagcacgggaccatgcaag

SEQ ID NO: 12 - DNA, 260 bp, Hepatitis B virus
ggacttctctcaattttctaggggaagctcccgcgtgtcctggccaaaattcgcagtccccaacctccaatcactcacaaacctcttgtccaccaatttgtcctggctatcgctggatgtgtctgcggcgttttatcatcttcctcttcatcctgctgctatgcctcatcttcttgttggttcttctggattaccaaggtatgttgcccgtttgtcctctacttccaggaacatcaactaccagcacgggaccatgcaag

SEQ ID NO: 13 - DNA, 260 bp, Hepatitis B virus
ggacttctctcaattttctagggggaactaccgtgtgtcttggccaaaattcgcagtccccaacctccaatcactcaccaacctcctgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcatcaaccaccagcacgggaccctgcaga

SEQ ID NO: 14 - DNA, 260 bp, Hepatitis B virus
ggacttctctcaattttctagggggagctcccgtgtgtcttggccaaaattcgcagtccccaatctccaatcactcaccaacctcttgtcctccaatttgtcctggctatcgctggatgtgtctgcggcgttttatcatcttcctcttcatcctgctgctatgcctcatcttcttgttggttcttctggactatcaaggtatgttgcccgtttgtcctctaattccaggatcatcaaccaccagtacgggaccctgccga

SEQ ID NO: 15 - DNA, 260 bp, Hepatitis B virus
ggacttctctcaattttctagggggactacccgggtgtcctggccaaaattcgcagtccccaacctccaatcacttaccaacctcctgtcctccaacttgtcctggctatcgttggatgtgtctgcggcgttttatcatcttcctcttcatcctgctgctatgcctcatcttcttgttggttcttctggactatcaaggtatgttgcccgtttgtcctctaattccaggatctacgaccaccagcacgggaccatgcaaa

SEQ ID NO: 16 - DNA, 260 bp, Hepatitis B virus
ggacttctctcaattttctagggggagtgcccgtgtgtcctggcctaaattcgcagtccccaacctccaatcactcaccaatctcctgtcctccaacttgtcctggctatcgctggatgtgtctgcggcgttttatcatattcctcttcatcctgctgctatgcctcatcttcttgttggttcttctggactatcaaggtatgttgcccgtttgtcctctgattccaggatcctcgaccaccagtacgggaccctgcaaa

SEQ ID NO: 17 - DNA, 260 bp, Hepatitis B virus
ggacttctctcaattttctaggggtaccacccgggtgtcctggccaaaattcgcagtccccaatctccaatcacttaccaacctcctgtcctccaacttgtcctggctatcgttggatgtgtctgcggcgttttatcatcttcctcttcatccggctgctatgccttcaccgtttgcctgtgtgtctggactatcaaggtatgttgcccgtgtgtcctctacttccaggatctacaaccaccagcacgggaccctgcaaa

SEQ ID NO: 18 - DNA, 260 bp, Hepatitis C virus
gtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcg

SEQ ID NO: 19 - DNA, 260 bp, Hepatitis C virus
gtagcgttgggttgcgaaaggccttgtggtactgcctgatagggtgcttgcgagtgccccgggaggtctcgtagaccgtgcaccatgagcacaaatcctaaacctcaaagaaaaaccaaaagaaacaccaaccgtcgcccacaagacgttaagtttccgggcggcggccagatcgttggcggagtatacttgttgccgcgcaggggccccaggttgggtgtgcgcgcgacaaggaagacttcggagcggtcccagccacg

SEQ ID NO: 20 - DNA, 260 bp, Hepatitis C virus
gtagtgttgggtcgcgaaaggccttgtggtactgcctgatagggtgcttgcgagtgccccgggaggtctcgtagaccgtgcaacatgagcacacttcctaaacctcaaagaaaaaccaaaagaaacaccatccgtcgcccacaggacgtcaagttcccgggtggcggacagatcgttggtggagtatacgtgttgccgcgcaggggcccacgattgggtgtgcgcgcgacgcgtaaaacttctgaacggtcacagcctcg

SEQ ID NO: 21 - DNA, 260 bp, Hepatitis C virus
gtagtgttgggtcgcgaaaggccttgtggtactgcctgatagggtgcttgcgagtgccccgggaggtctcgtagaccgtgcaccatgagcacgaatcctaaacctcaaagaaaaaccaaacgtaacaccaaccgccgcccaatggacgttaagttcccgggtggtggccagatcgttggcggagtttacttgttgccgcgcaggggccccagattgggtgtgcgcgcgactcggaagacttcggagcggtcgcaacctcg

SEQ ID NO: 22 - DNA, 260 bp, Hepatitis C virus
gtagtgttgggtcgcgaaaggccttgtggtactgcctgatagggtgcttgcgagtgccccgggaggtctcgtagaccgtgcaccatgagcacgaatcctaaacctcaaagaaaaaccaaaagaaacaccaaccgccgcccacaggacgtcaagttcccgggcggtggtcagatcgttggtggagtttacttgttgccgcgcaggggccctaggttgggtgtgcgcgcgactcggaagacttcagaacggtcgcaaccccg

SEQ ID NO: 23 - DNA, 260 bp, Hepatitis C virus
gtagcgttgggttgcgaaaggccttgtggtactgcctgatagggtgcttgcgagtgccccgggaggtctcgtagaccgtgcaacatgagcacacttcctaaacctcaaagaaaaaccaaaagaaacaccaaccgtcgcccaatggacgtcaagttcccgggcggcggccagatcgttggtggagtttacttgctgccgcgcaggggcccacgattgggtgtgcgcgccacaagaaagacatccgagcgatcccagcccag

SEQ ID NO: 24 - DNA, 84 bp, Homo sapiens
catgaagatcctcaccgagcgcggctacagcttcaccaccacggccgsgcgggaaatcgtgcgtgacattaaggagaagctgtg

(19) United States
(12) Patent Application Publication
(10) Pub. No.: US 2016/0017442 A1
(43) Pub. Date: Jan. 21, 2016

(54) Multiplex Real Time PCR Testing Kit for the Simultaneous Detection of Hepatitis Virus

(71) Applicants: INDIAN COUNCIL OF MEDICAL RESEARCH, Ansari Nagar, New Delhi (IN); KING GEORGE'S MEDICAL UNIVERSITY, Lucknow, Uttar Pradesh (IN)

(72) Inventors: Amita Jain, Lucknow (IN); Shantanu Prakash, Lucknow (IN)

(73) Assignees: Indian Council of Medical Research, New Delhi (IN); Kings George's Medical University, Lucknow (IN)

(21) Appl. No.: 14/772,821

(22) PCT Filed: Mar. 4, 2014
(86) PCT No.: PCT/IN2014/000143

§ 371 Date: Sep. 4, 2015

(30) Foreign Application Priority Data
Mar. 5, 2013 (IN) 622/DEL/2013

(51) Int. Cl. C12Q 1/70

(52) U.S. Cl. CPC C12Q 1/706; C12Q 2600/16

(57) ABSTRACT

Provided herein are primers and probes for the detection of Hepatitis B virus and Hepatitis C virus in a sample, a reaction mixture for multiplex real time PCR for the simultaneous detection and quantitation of Hepatitis B and C and a test kit based on multiplex real time PCR for the simultaneous detection and quantitation of Hepatitis B and C.

### FIELD OF THE INVENTION

This invention relates to probes and primers for the simultaneous detection of Hepatitis virus and a reaction mixture for multiplex PCR which enables the equal intensity detection of hepatitis B and C viruses individually.

This invention further relates to in vitro diagnostic assays for the detection of Hepatitis virus in human plasma or serum sample. More particularly, the invention relates to a highly sensitive, specific and cost effective testing kit based on multiplex PCR, which enables simultaneous detection of hepatitis B virus and hepatitis C virus. The test kit essentially comprises probes & primers for the detection of hepatitis B virus and hepatitis C virus while an internal control for checking the validity of the reaction. The kit advantageously helps in detecting all the genotypes of Hepatitis B and C virus and also the provided probes and primers are efficient enough to detect sample with low copy number viruses.

Further, the invention extends to provide a reaction mixture for the multiplex PCR which enables the equal intensity detection of hepatitis B and C viruses individually.

### BACKGROUND OF THE INVENTION

HBV and HCV infections are major public health problems and leading causes of liver diseases (acute and chronic hepatitis), leading to cirrhosis and hepatocellular carcinoma. Recent studies have shown that throughout the world number of people infected with hepatitis B virus (HBV) and Hepatitis C virus (HCV) is amounting to about 350 million and 170 million respectively.

HCV is an enveloped virus belonging to the Flaviviridae family. The viral genome is a linear, positive-stranded RNA molecule of 9600 nucleotides that contains a single open reading frame which codes for a polyprotein of 3000 amino acids. The amino-terminal portion of the viral. RNA encodes for the structural proteins (C, M, E1 and E2), followed by nonstructural proteins (NS1, NS2, NS3, NS4A, NS4B, NS5A, and NS5B). The HCV turnover rate can be quite high with replication ranging between 10¹⁰ to 10ⁿ virions per day, and a predicted viral half-life of 2 to 3 hours. The rapid viral replication and lack of error proofreading by the viral RNA polymerase are reasons why the HCV RNA genome mutates frequently. There are six known genotypes (numbered 1 to 6) and more than 50 subtypes (e.g., 1a, 1b, 2a etc.)

HBV is classified in the family Hepadnaviridae. It circulates as eight distinct genotypes, designated A to H, but it is controversial as to whether the outcome of the infection is influenced by the genotype. HBV has a double-stranded DNA genome of approximately 3200 base pairs organized into four partially overlapping open reading frames, which encode the envelope, core (precore/core), polymerase and X proteins. The envelope proteins are surface glycoproteins collectively designated, as hepatitis B surface antigen (HBsAg). It serves as a marker for active infection and infectivity. The core open reading frame encodes a polypeptide that is expressed as either the hepatitis B e antigen (HBeAg) or the viral capsid protein (HBcAg). The presence of detectable HBeAg in serum or plasma is associated with high levels of HBV replication, greater infectivity and an increased risk of hepatic fibrosis. Mutations in the core promoter and pre-core regions result in decreased levels or an absence of detectable HBeAg in the serum, but this may not alter the squeal of chronic infection.

HBV and HCV share common modes of transmission and as a result, combined HBV and HCV infection is becoming frequent, especially HBV and HCV co-infection is not uncommon in geographic areas where a high endemic level of both infections is reported, such as India, South-Asia and Mediterranean. In general, the prevalence of HCV co-infection is around 10-20% in patients with chronic HBV infection. Similarly total 2-10% of anti-HCV-positive patients are positive for HBV infection as well. The exact prevalence of dual HBV and HCV infection is not known. Most of the published statistics focused on highly selective and limited populations such as injection drug users (IDU, 42.5%), patients on hemodialysis (3.7%), patients undergoing organ transplantation (8%), HIV-positive individuals (66%) and beta-thalassemia patients (10%), etc. which means that these are the high risk population for HBV and HCV co-infection.

The increasing incidence of HBV and HCV co-infection raises the demand for a highly sensitive, specific and cost-effective test having less turnout time for simultaneous detection of HBV and HCV. The PCR based assays for the simultaneous detection of HBV and HCV nucleic acids in the serum or plasma of an infected subject may provide an advantage.

The currently used methods for the diagnosis of HBV and HCV is based on ELISA (Enzyme Linked immune sorbent assay) for detecting the serum markers such as, HbeAg, HbsAg, anti-HBdgM, anti-HBcIgM, anti-HBe, anti-HBs, anti-HBcIgGs, for HBV and HCV total antibody for HCV. Since ELISA based methods are not sensitive and reliable, there is a need to look for a method which can give a sensitive, reliable and cost effective diagnosis. There are a number of assays for HBV and HCV detection in uniplex reaction and few have HBV, HCV & HIV multiplex. These assays do not have internal quality control system. Moreover due to genotypic diversity many of the assays have limitations in picking up all the existing genotypes. The currently available test for the detection of HBV and HCV do not have any feature which can provide assurance that the performed test on the same has run successfully or not; as a result causes perplexity in determining the actual diseased state of a patient. This directly affects the result as well as the treatment of the patients, as the treatment is highly dependent on viral load.

KR2012001874 discloses a detection method and kit for detecting HBV (hepatitis B virus) in a test sample. However, the test does not detect the presence of Hepatitis C virus.

U.S. Pat. No. 5,830,711 (Barany et al 1998) describes a method for distinguishing a first nucleotide sequence which differs by at least a single base from a second nucleotide sequence by using ligase chain reaction (LCR) utilizing the thermophilic DNA ligase from Thermus aquaticus to detect a target DNA sequence.

The detection methods described in the prior art give variable results with respect to non-specificity, poor amplification signal and non reproducibility. It is therefore desirable to provide a method of detection of the target nucleic acid in a sample using ligase mediated amplification reaction that overcomes the above-mentioned disadvantages.

Therefore, there is a need to provide a highly sensitive, specific and cost-effective testing kit which enables the simultaneous detection of hepatitis B virus and hepatitis C virus overcoming the problem of missing any case i.e. which can help in detecting all the genotypes of Hepatitis B and C virus.

There is also a need of a test kit which helps in detecting the samples infected with low copy number viruses.

Further, there is also a need of a test kit which comprises an internal control which enables the checking of the validity of the reaction so as to be ensured that the test has run successfully on the sample.

Also, there is the requirement of a reaction mixture for the multiplex PCR which enables the equal intensity of detection of Hepatitis B and C viruses separately i.e. the higher presence of one type of hepatitis virus does not affect the detection of the other hepatitis virus in the sample.

### OBJECTS OF THE INVENTION

It is there an object of this invention to propose probes and primers, which are capable of detecting Hepatitis B and Hepatitis C virus simultaneously.

It is a further object of this invention to propose probes and primers, which are highly sensitive and specific for the detection of Hepatitis B and Hepatitis C virus simultaneously.

Another object of this invention is to propose probes and primers, which are capable of detecting all the genotypes of Hepatitis B and C virus.

Yet another object of this invention is of this invention to propose probes and primers, which help in attracting the sample with low copy number viruses of Hepatitis B and C viruses.

A further object of this invention is to propose probes and primers, which are capable of checking the validity of the reaction to ensure that the test has run successfully.

A still further object of the invention is to propose probes and primers, which enable the equal intensity of detection of Hepatitis B and C virus separately.

These and other objects and advantages of the invention will be apparent from the ensuing description with the help of the accompanying drawings.

### BRIEF DESCRIPTION OF THE ACCOMPANYING DRAWINGS

FIG. 1 shows the alignment of HBV primers and probe with S region of all eight genotype of HBV is shown.

FIG. 2 shows alignment of HCV primers and probe with 5′NTR region of all six genotype of HCV.

FIG. 3 shows alignment of internal control primers and probe with Human β-actin gene.

FIG. 4 shows a Real time plot of HBV positive samples using SEQ ID NO. 1, 2 & 3 red showing HBV amplification and green line showing No. Template Control.

FIG. 5 shows a Real time plot of HCV positive samples using SEQ ID NO. 4, 5 & 6 red showing HCV amplification and green line showing NTC.

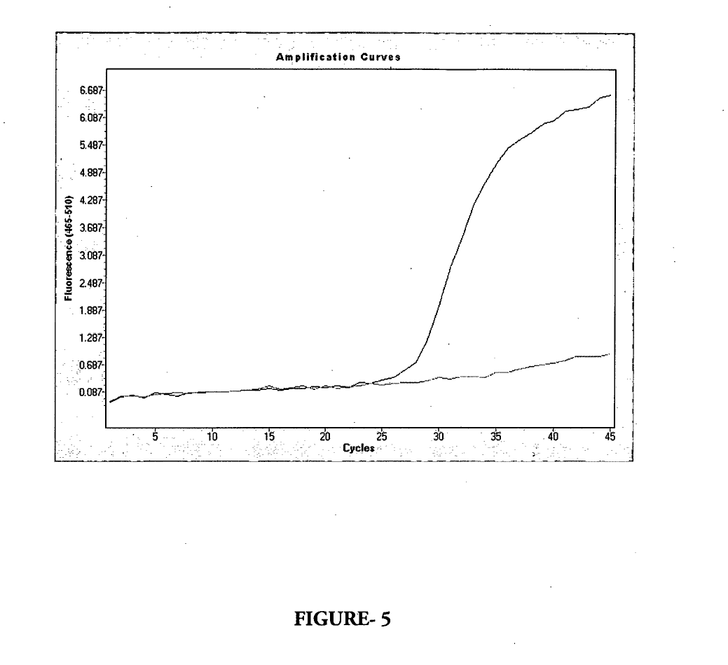

FIG. 6 shows a Real time plot of β-actin positive samples using SEQ ID No. 7, 8 & 9 red showing Human β-actin amplification and green line showing NTC.

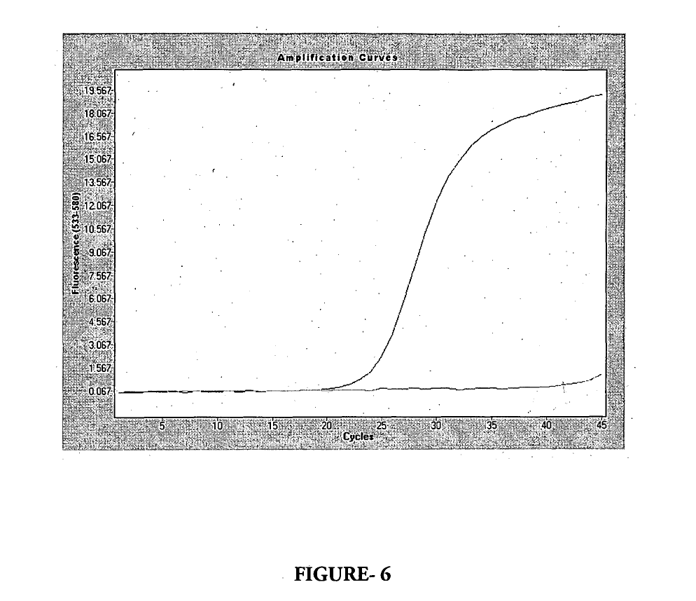

FIG. 7 shows a Real time plot of HBV positive and HCV negative sample in a multiplex reaction using SEQ ID No. 1 to 9 (a) HBV positive with NTC (filter 618-660), (b) HCV negative with NTC (filter 465-510) and (c) Human β-actin with NTC (filter 533-580)

FIG. 8 shows a Real time plot of HBV negative and HCV positive sample in a multiplex reaction using SEQ ID No. 1 to 9 (a) HBV negative with NTC (filter 618-660), (b) HCV negative with NTC (filter 465-510) and (c) Human β-actin with NTC (filter 533-580)

FIG. 9 shows a Real time plot of HBV negative and HCV positive sample in a multiplex reaction using SEQ ID No. 1 to 9 (a) HBV negative with NTC (filter 618-660), (b) HCV negative with NTC (filter 465-510) and (c) Human β-actin with NTC (filter 533-580)

FIG. 10 shows a Real time plot of HBV negative and HCV co-infection sample in a multiplex reaction using SEQ ID No. 1 to 9 (a) HBV positive with NTC (filter 618-660), (b) HCV positive with NTC (filter 465-510) and (c) Human β-actin with NTC (filter 533-580)

FIG. 11 shows a Real time plot of for comparison between Cp of company kit with the kit according to the invention in a HBV positive sample (a) HBV positive with NTC (filter 465-510) using company kit, (b) HBV positive with NTC (filter 618-660) and (c) Human β-actin with NTC (filter 533-580)

FIG. 12 shows a Real time plot of for comparison between Cp of company kit with the kit according to the invention in a HCV positive sample (a) HCV positive with NTC (filter 465-510) using company kit, (b) HCV positive with NTC (filter 465-510) and (c) Human β-actin with NTC (filter 533-580)

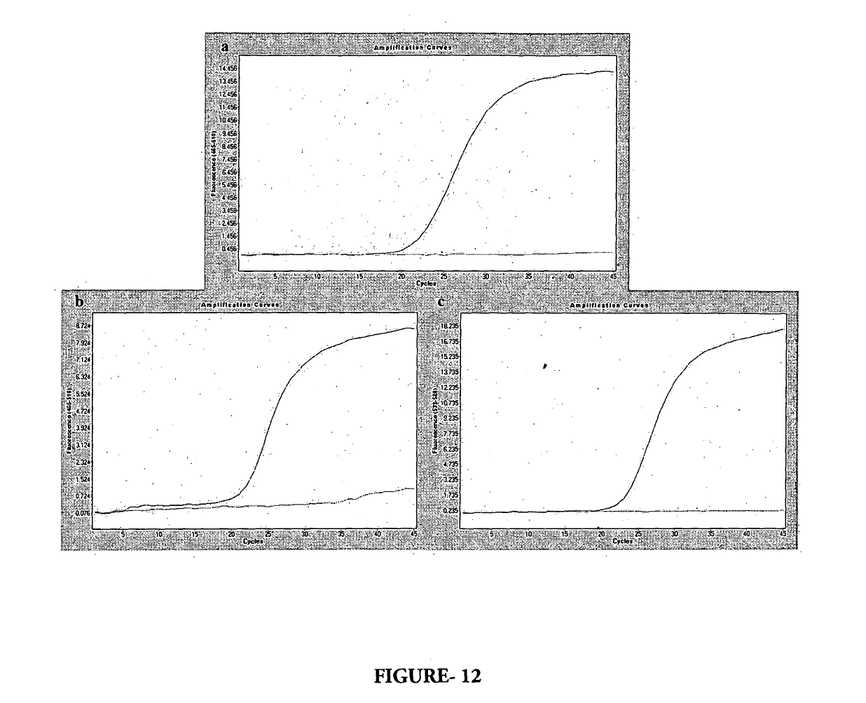

### SUMMARY OF THE INVENTION

This invention relates to probes and primers multiplex real time PCR for the detection of Hepatitis B virus and Hepatitis C virus. This invention further relates to a reaction mixture for the multiplex PCR which enables the equal intensity detection of hepatitis B and C viruses individually i.e. the higher presence of one type of hepatitis virus does not affect the detection of the other hepatitis virus in the same.

The invention, further relates to in vitro diagnostic assays for the simultaneous detection of Hepatitis B and C Virus with low copy number up to 60 IU/ml and 20 IU/ml respectively. The invention further relates to a test kit for the simultaneous detection and quantitation of Hepatitis B virus and Hepatitis C virus.

The test kit essentially comprises the probes & primers for the detection of hepatitis B virus and hepatitis C virus with an internal control for checking the validity of the reaction. The kit advantageously helps in detecting all the genotypes of Hepatitis B and C virus and also the provided probes and primers are efficient enough to detect sample with low copy number viruses.

In the present embodiment, the invention has been described with reference to human plasma or serum sample, however such description should not be considered as restricting the scope of the present invention. Further it would be possible for a person skilled in the art to practice the present invention considering other sample for the detection of hepatitis B and C virus without departing from the scope of the present invention.

### DETAILED DESCRIPTION OF THE INVENTION

According to this invention is provided primers and probes for the detection of Hepatitis B and Hepatitis C virus in a sample.

The invention provides primers and probes for the detection of Hepatitis B virus in a sample comprising:

a) a nucleic acid molecule that encodes a nucleotide sequence of SEQ ID NO: 1 or a part of it or a nucleotide having at least 90% sequence identity with said SEQ ID NO:1, wherein said nucleotide sequence of SEQ ID NO: 1 represents forward primer to amplify hepatitis B virus;
b) a nucleic acid molecule that encodes a nucleotide sequence of SEQ ID NO: 2 or a part of it or a nucleotide having at least 90% sequence identity with said SEQ ID NO:2, wherein said nucleotide sequence of SEQ ID NO: 2 represents reverse primer to amplify hepatitis B virus;
c) a nucleic acid molecule that encodes a nucleotide sequence of SEQ ID NO: 3 or a part of it or a nucleotide having at least 90% sequence identity with said SEQ ID NO:3, wherein said nucleotide sequence of SEQ ID NO: 3 represents probes to detect hepatitis B virus;
and wherein said nucleotide sequences' enables the high detection of all genotypes of Hepatitis B and C virus.

The nucleotide sequences enables detection of Hepatitis B and C virus present in low copy number up to 60 IU/ml and 20 IU/ml respectively.

The invention also provides primers and probes for the detection of Hepatitis C virus in a sample comprising:

a) a nucleic acid molecule that encodes a nucleotide sequence of SEQ ID NO:4 or a part of it or a nucleotide having at least 90% sequence identity with said SEQ ID NO:4, wherein said nucleotide sequence of SEQ ID NO:4 represents forward primer to amplify hepatitis C virus;
b) a nucleic acid molecule that encodes a nucleotide sequence of SEQ ID NO:5 or a part of it or a nucleotide having at least 90% sequence identity with said SEQ ID NO:5, wherein said nucleotide sequence of SEQ ID NO:5 represents reverse primer to amplify hepatitis C virus;
c) a nucleic acid molecule that encodes a nucleotide sequence of SEQ ID NO:6 or a part of it or a nucleotide having at least 90% sequence identity with said SEQ ID NO:6, wherein said nucleotide sequence of SEQ ID NO:6, wherein said nucleotide sequence of SEQ ID NO:6 represents probes for the detection of heptatitis C virus;
and wherein said nucleotide ‘sequences enables the high detection of all genotypes of Hepatitis C and wherein said nucleotide sequences enables detection of virus present in low copy number up to 20 IU/ml.

The invention’ further provides a reaction mixture for multiplex real time PCR for the simultaneous detection and quantitation of Hepatitis virus comprising:

a) Nuclease free water present in an amount of 0.950
b) RT-Buffer present in an amount of 6.25 μl
c) HCV Primer Forward (10 pm) present in an amount of 0.35 μl
d) HCV Primer Reverse (10 pm) present in an amount of 0.35 μl
e) HBV Primer Forward (10 pm) present in an amount of 0.35 μl
f) HBV Primer Reverse (10 pm) present in an amount of 0.35 μl
g) β-actin Primer Forward (10 pm) present in an amount of 0.20 μl
h) β-actin Primer Reverse (10 pm) present in an amount of 0.20 μl
i) HCV Probe (10 pm) present in an amount of 0.175 μl
j) HBV Probe (10 pm) present in an amount of 0.175 μl
k) B-actin Probe (10 pm) present in an amount of 0.15 μl
l) R.T. Enzyme present in an amount of 0.5 μl
m) Template present in an amount of 7.5 μl
wherein said reaction mixture enables equal intensity detection of all genotypes of Heatitis B and C virus.

The multiplex real-time polymerase chain reaction for HBV and HCV is provided with Human β-actin gene as internal control.

According to this invention is further provided a test kit based on multiplex real time. PCR for the simultaneous detection and quantitation of Hepatitis virus, said kit comprising at least one nucleotide sequence selected from the group comprising of:

a) a nucleic acid molecule that encodes a nucleotide sequence of SEQ ID NO: 1 or a part of it or a nucleotide having 90% sequence identity with said SEQ ID NO:1;
b) a nucleic acid molecule that encodes a nucleotide sequence of SEQ ID NO: 2 or a part of it or a nucleotide having 90% sequence identity with said SEQ ID NO:2;
c) a nucleic acid molecule that encodes a nucleotide sequence of SEQ ID NO: 3 or a part of it or a nucleotide having 90% sequence identity with said SEQ ID NO:3;
d) a nucleic acid molecule that encodes a nucleotide sequence of SEQ ID NO: 4 or a part of it or a nucleotide having 90% sequence identity with said SEQ ID NO:4;
e) a nucleic acid molecule that encodes a nucleotide sequence of SEQ ID NO: 5 or a part of it or a nucleotide having 90% sequence identity with said SEQ ID NO:5;
f) a nucleic acid molecule that encodes a nucleotide sequence of SEQ ID NO: 6 or a part of it or a nucleotide having 90% sequence identity with said SEQ ID NO:6;
g) an internal control gene;
wherein said nucleotide sequences enable high detection of all genotypes of Hepatitis B and C virus.

The nucleotide sequences enable detection of Hepatitis B and C virus present in low copy number up to 60 IU/ml and 20 IU/ml respectively.

### Designing the Primers

### HBV

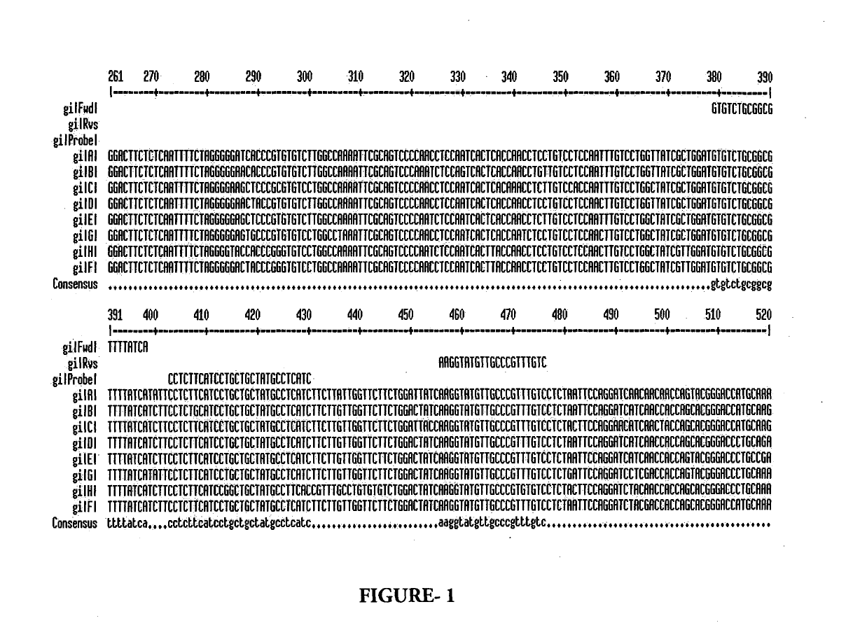

Sequences representing all eight HBV genotypes (A-H) accession no. (genotype A: AP007263.1, genotype B: AB602818.1, genotype C: AB644286.1, genotype D: FJ692536.2, genotype E: AP 007262.1, genotype F: AF 288628.1, genotype G: AP007264.1, genotype H: AB516395.1) were downloaded from the GenBank nucleotide database and aligned using the program multalin. A highly is conserved region of the S gene is selected for the design of real-time PCR primers and probe. All eight HBV genotypes which we studied are presented in FIG. 1. A total of six degeneracies are done to make the primers and probe suitable for all the genotypes. The probe is tagged with Cy5 as a reporter and IAbRQSp as a quencher at 3′ end. The primers and probes sequences for HBV are mentioned in TABLE 1:

TABLE 1

| Sequence | | Oligonuceotide | | Product |
|---|---|---|---|---|
| ID | Polarity | sequence 5′-3′ | Length | size |
| 1. HBV | Forward | GTGTCTGCGGCGTTTTATCA | 20 | 98 bp |
| Fwd | | | | |
| 2. HBV | Reverse | GACAMACGGGCAACATACCTT | 21 | |
| Rvs | | | | |
| 3. HBV | | /5Cy5/CCTCTKCATCCKGCTGCTATGC | 28 | |
| Probe | | CTY | | |
| | | MWC/3IAbRQSp/ | | |

### HCV

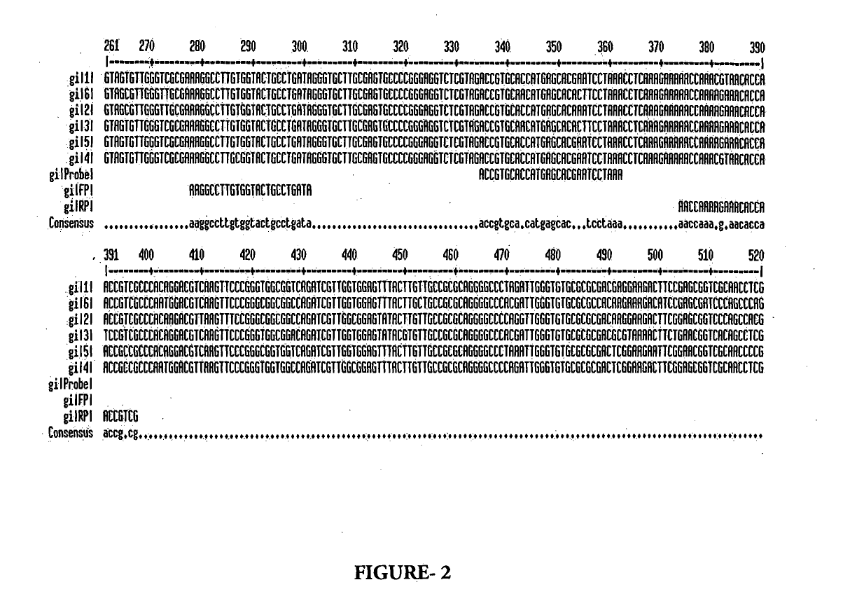

Nucleotide sequence of the 5′ NC region of the HCV genome from all the six genotypes (genotype 1: NC_{—}004102.1, genotype 2: NC_{—}009823.1, genotype 3: NC_{—}009824.1, genotype 4: NC_{—}009825.1, genotype 5: NC_{—}009826.1, genotype 6: NC_{—}009827.1) was analyzed from large number of isolates obtained throughout the world. The sequences of all the genotypes were aligned using multalin to find conserved sequence of HCV and primers & probe was designed. An alignment of primers & probe with nucleotide sequence of the 5′ Non translated region from the all six HCV genotypes which we studied is presented in FIG. 2. A total of six degeneracies are done to make the primers and probe suitable for all the genotypes. The probe is tagged with FAM as a reporter and IABkFQ as a quencher at 3′ end. The primers and probe sequences for HCV are mentioned in table 2.

TABLE 2

| Sequence | | Oligonuceotide | | Product |
|---|---|---|---|---|
| ID | Polarity | sequence 5′-3′ | Length | size |
| 4. HBV | Forward | AAGGCCTTGTGGTACTGCCTGATA | 24 | 120 bp |
| Fwd | | | | |
| 5. HCV | Reverse | CGACGGTTGGTGTTWCKTTTGGTT | 24 | |
| Rvs | | | | |
| 6. HCV | | 5′-56- | 28 | |
| Probe | | FAM/ACCGTGCAMCATGAGCACRMWT | | |
| | | CCTAAA/3IABkFQ/-3′ | | |

### Human β-Actin

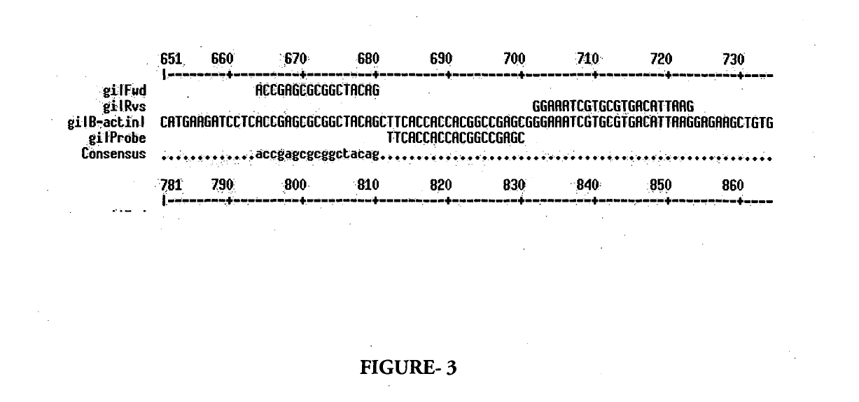

β-actin (gene name ACTB) is one of six different actin proteins. Actins are highly conserved proteins that are involved in cell motility, structure, and integrity. Actin is a major constituent of the contractile apparatus and one of the two non-muscle cytoskeletal actins. Housekeeping gene is typically a constitutive gene that is required for the maintenance of basic cellular functions and expressed in all cells of an organism. Human β-actin gene is expressed at relatively constant levels. An alignment of primers & probe with nucleotide sequence of β-actin, using multalin is shown in FIG. 3. The probe designed is tagged with HEX as reporter and double quenched with ZEN and IABkFQ. The primers and probe sequences for Human (β-actin are mentioned in table 3

TABLE 3

| Sequence | | Oligonuceotide | | Product |
|---|---|---|---|---|
| ID | Polarity | sequence 5′-3′ | Length | size |
| 7. β-actin | Forward | ACCGAGCGCGGCTACAG | 17 | 60 bp |
| Fwd | | | | |
| 8. B-actin | Reverse | CTTAATGTCACGCACGATTTCG | 22 | |
| Rvs | | | | |
| 9. β-Actin | | 5′-/ | 19 | |
| Probe | | 5HEX/TTCACCACC/ZEN/ACGGCCGA | | |
| | | GC/3IA BKFQ/-3′ | | |

### Testing the Probes and Primers:

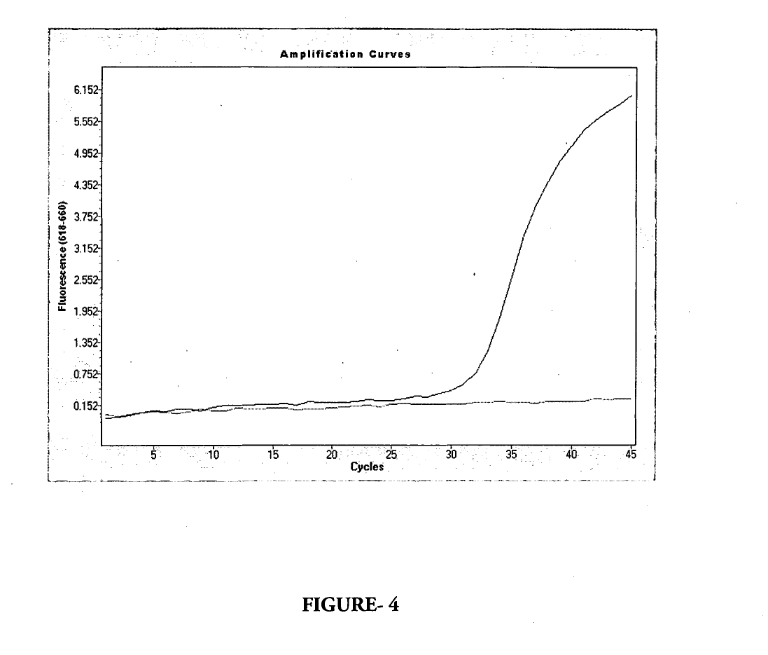

A batch of 220 samples was tested for HBV and HCV by currently designed primer and probes both in uniplex and multiplex. Results are shown in table no. 4. Real time plot of HBV, HCV and Human β-actin positive samples using SEQ ID No. 1, 2 & 3 in uniplex reaction are shown in FIGS. 4,5 & 6 respectively, red line showing amplification in sample and green line showing no template control.

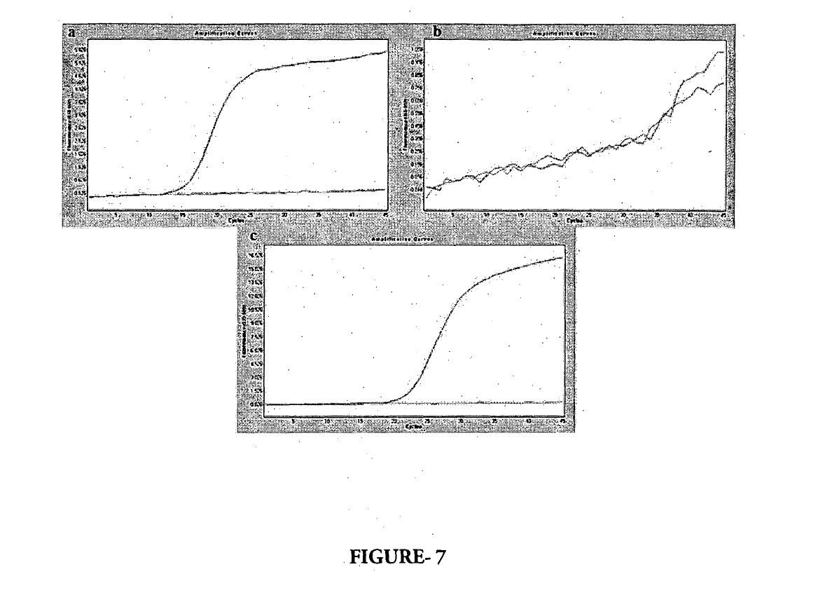

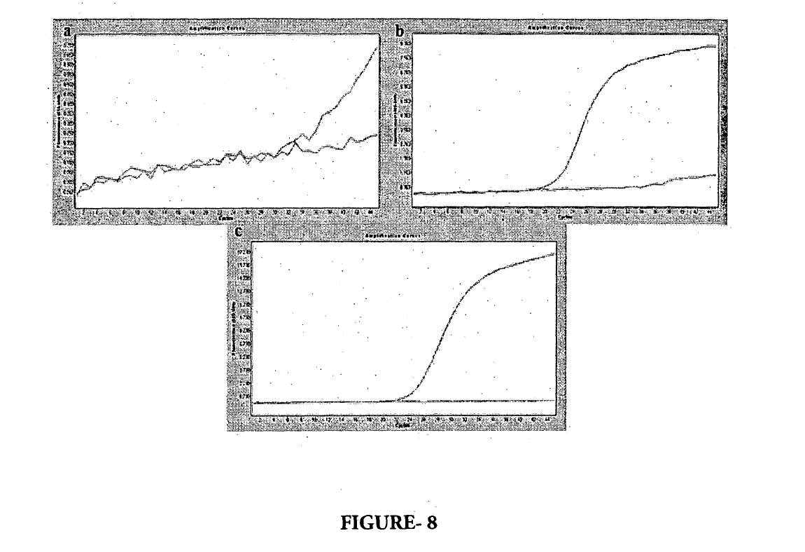

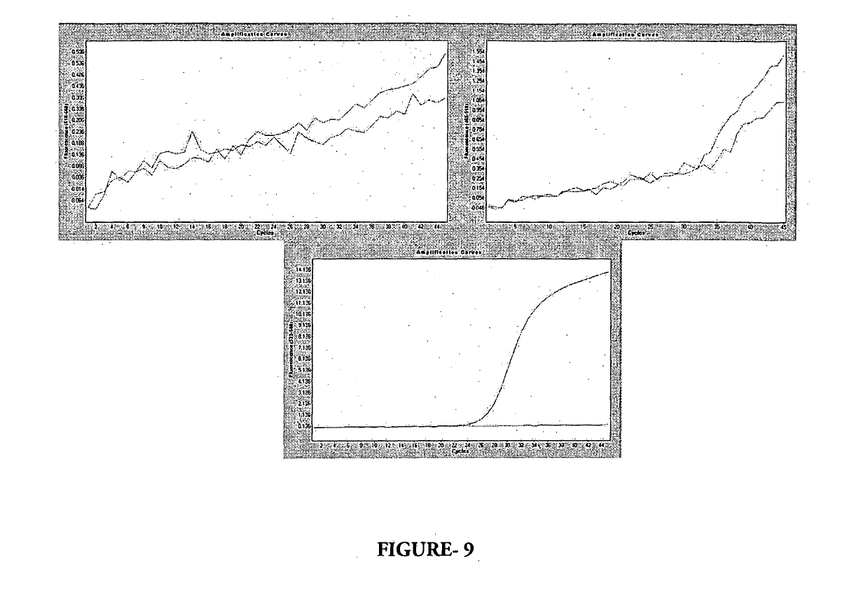

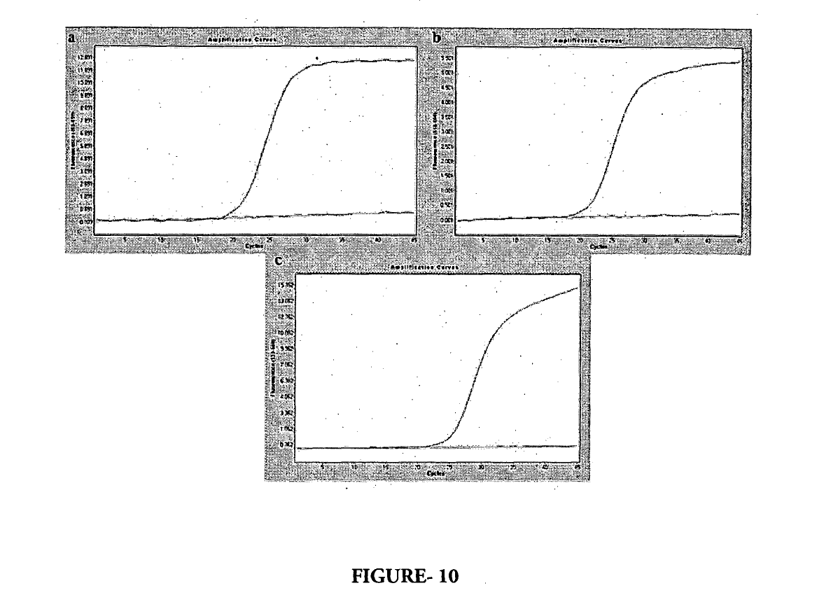

Same lot of 220 samples were tested in multiplex reactions using the same primer and probe (SEQ ID No. 1, 2 & 3) as shown in FIGS. 7,8,9 & 10. Red line in figure is showing amplification & green line is no template control. The results are shown in table 4. FIG. 7 shows Test plot of HBV positive and HCV negative sample, FIG. 8 shows test plot of HBV negative and HCV positive sample, FIG. 9 shows plot of HBV negative and HCV negative samples, while FIG. 10 is showing plot of sample positive for both HBV and HCV were made.

Total 48 samples tested positive for HBV while 72 tested positive for HCV. Human β-actin gene was amplified in all the 220 samples in both uniplex and multiplex reaction. Human β-actin gene had mean Cp of 24.65.

Verification of test results using the probes and primers was done by conventional and real time PCR, using commercial kits:

Comparison with Commercial Kit

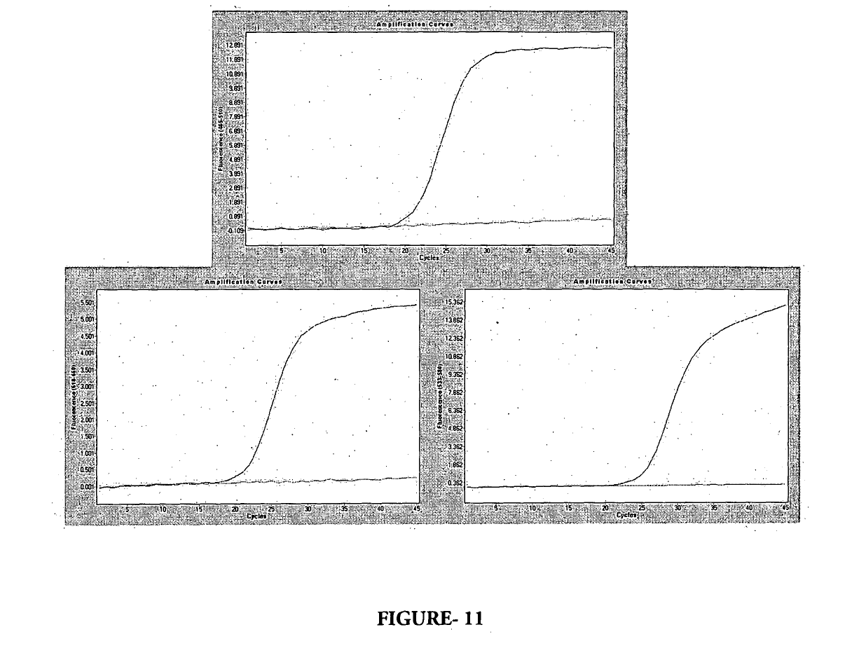

All the samples were tested with 2 commercial kits, used for detection of HBV & HCV in uniplex. There is significant variation between the results of HCV with the probes and primers according to the instant invention and the company kit (table 4). There was no significant difference found between the Cp of commercial kit and our designed probes and primers. FIGS. 11 & 12 are showing real time plot of a sample tested for both HBV & HCV with commercial kit and our kit (multiplex reaction). Red line is showing amplification and green line showing no template control.

Comparison with Conventional PCR

All 220 samples were tested in a conventional uniplex PCR setting using published primers for HBV (Olioso D et al) and HCV (Bukh et al).

Results were 100% concordant (table 4).

TABLE 4
Comparative results of claimed primers and probes, with
commercially available kit and published primers (n = 220)

| | Uniplex | Multiplex | | |
|---|---|---|---|---|
| | with | with our | | |
| | our newly | newly | | |
| | designee | designed | | |
| | and Probes | Probes and | Commercial | Conventional |
| | Primers | Primers | kit | PCR |
| HBV (positives/ | 48/220 | 48/220 | 48/220 | 48/220 |
| number tested) | | | | (Olioso |
| | | | | D et al) |
| HCV (positives/ | 72/220 | 72/220 | 67/220 | 72/220 |
| number tested) | | | | (Bukh et al) |
| Human β-actin | 220/220 | 220/220 | NA | NA |
| (positives/ | | | | |
| number tested) | | | | |

### Genotype Detection Using Newly Designed Probes and Primers

All the positive samples for HBV & HCV were sequenced using ABI 3130 sequencer using the same primer used for conventional. PCR. Sequencing results showed genotypes A, B, D & H among HBV positive samples and genotype 1, 3, 5 & 6 in HCV positive samples and all of them were picked strongly by probes and primers according to the invention.

### Testing the Influence of High Viral Load of One Virus Over the Amplification of Low Viral Copy Number of Other Virus:

Another set of experiment was done to ensure that there is no effect of high viral load of one virus over the amplification of low copy number of the other virus in multiplex reaction. Sample with high viral load of HCV (4.46×10⁷) was mixed with 10 fold dilution of HBV sample starting with 10⁷ viral load (2.89×10⁷) and it was seen that there was no such effect on HBV detection and reaction was able to pick up to 60 IU/ml copy numbers of HBV. The same experiment was also done for HCV and the results were similar i.e. up to 20 IU/ml copy numbers were picked up. It was also assured that the detection of both the viruses is not affected by simultaneous detection of Human β-actin gene.

The invention also provides a reaction mixture comprising following components:
a) Nuclease free water present in an amount of 0.95 μl.
b) RT-Buffer present in an amount ranging from 6.25 μl.
c) HCV Primer Forward (10 pm) present in an amount ranging from 0.35 μl.
d) HCV Primer. Reverse (10 pm) present in an amount ranging from 0.35 μl.
e) HBV Primer Forward (10 pm) present in an amount ranging from 0.35 μl.
f) HBV Primer Reverse (10 pm) present in an amount ranging from 0.35 μl.
g) β-actin Primer Forward (10 pm) present in an amount ranging from 0.20 μl.
h) β-actin Primer Reverse (10 pm) present in an amount ranging from 0.20 μl
i) HCV Probe (10 pm) present in an amount ranging from
j) 0.175 μl
k) HBV Probe (10 pm) present in an amount ranging from
l) 0.175 μl
m) β-actin Probe (10 pm) present in an amount ranging from
n) 0.15 μl
o) R.T. Enzyme present in an amount ranging from 0.5 μl
p) Template present in an amount ranging from 7.5 μl

The reaction mixture advantageously enables equal intensity detection of all the genotypes of Hepatitis B and C virus.

1. A primer for the detection of Hepatitis B virus in a sample, comprising:
a) a nucleic acid molecule that encodes a nucleotide sequence of SEQ ID NO: 1 or a part of it or a nucleotide having at least 90% sequence identity with said SEQ ID NO: 1, wherein said nucleotide sequence of SEQ ID NO:
1 represents forward primer to amplify hepatitis B virus;
b) a nucleic acid molecule that encodes a nucleotide sequence of SEQ ID NO: 2 or a part of it or a nucleotide having at least 90% sequence identity with said SEQ ID NO:2, wherein said nucleotide sequence of SEQ ID NO:
2 represents reverse primer to amplify hepatitis B virus; and/or
c) a nucleic acid molecule that encodes a nucleotide sequence of SEQ ID NO: 3 or a part of it or a nucleotide having at least 90% sequence identity with said SEQ ID NO:3, wherein said nucleotide sequence of SEQ ID NO:
3 represents probes to detect hepatitis B virus.

2. The primer as claimed in claim 1, wherein said nucleotide sequences enables the high detection of all genotypes of Hepatitis B and the detection of virus present in low copy number up to 60 IU/ml.

3. The primer as claimed in claim 1, wherein said primer is sensitive to detect Hepatitis B with a viral load of 20 IU/ml.

4. A primer for the detection of Hepatitis C virus in a sample comprising:
a) a nucleic acid molecule that encodes a nucleotide sequence of SEQ ID NO: 4 or a part of it or a nucleotide having at least 90% sequence identity with said SEQ ID NO:4, wherein said nucleotide sequence of SEQ ID NO:
4 represents forward primer to amplify hepatitis C virus;
b) a nucleic acid molecule that encodes a nucleotide sequence of SEQ ID NO: 5 or a part of it or a nucleotide having at least 90% sequence identity with said SEQ ID NO: 5, wherein said nucleotide sequence of SEQ ID NO:
5 represents reverse primer to amplify hepatitis C virus; and/or
c) a nucleic acid molecule that encodes a nucleotide sequence of SEQ ID NO: 6 or a part of it or a nucleotide having at least 90% sequence identity with said SEQ ID NO:6, wherein said nucleotide sequence of SEQ ID NO:
6 represents probes to detect hepatitis C virus.

5. The primer as claimed in claim 4, wherein said nucleotide sequences enables the high detection of all genotypes of Hepatitis C virus and the detection of virus present in low copy number up to 20 IU/ml.

6. The primer as claimed in claim 4, wherein said primer is sensitive to detect Hepatitis C with a viral load of 20 IU/ml.

7. A reaction mixture for multiplex real time PCR for the simultaneous detection and quantitation of Hepatitis virus comprising:
a) Nuclease free water present in an amount of 0.95 μl;
b) Reverse transcript buffer present in an amount of 6.25 μl;
c) HCV Primer Forward present in an amount of 0.35 μl;
d) HCV Primer Reverse present in an amount of 0.35 μl;
e) HBV Primer Forward present in an amount of 0.35 μl;
f) HBV Primer Reverse present in an amount of 0.35 μl;
g) β-actin Primer Forward present in an amount of 0.20 μl;
h) β-actin Primer Reverse present in an amount of 0.20 μl;
i) HCV Probe present in an amount of 0.175 μl;
j) HBV Probe present in an amount of 0.175 μl;
k) β-actin Probe present in an amount of 0.150 μl;
l) Reverse Transcriptase present in an amount of 0.5 μl; and
m) Template present in an amount of 7.5 μl.

8. The reaction mixture as claimed in claim 7, wherein the HCV Probe comprises FAM and IABkFQ.

9. The reaction mixture as claimed in claim 7, wherein the HBV Probe comprises Cy5 and IAbRQSp.

10. The reaction mixture as claimed in claim 8, wherein the β-actin Probe comprises HEX and IABkFQ.

11. A test kit based on multiplex real time PCR for the simultaneous detection and quantitation of Hepatitis virus, said kit comprises one or more of:
a) a nucleic acid molecule that encodes a nucleotide sequence of SEQ ID NO: 1 or a part of it or a nucleotide having 90% sequence identity with said SEQ ID NO: 1;
b) a nucleic acid molecule that encodes a nucleotide sequence of SEQ ID NO: 2 or a part of it or a nucleotide having 90% sequence identity with said SEQ ID NO: 2;
c) a nucleic acid molecule that encodes a nucleotide sequence of SEQ ID NO: 3 or a part of it or a nucleotide having 90% sequence identity with said SEQ ID NO:3;
d) a nucleic acid molecule that encodes a nucleotide sequence of SEQ ID NO: 4 or a part of it or a nucleotide having 90% sequence identity with said SEQ ID NO:4;
e) a nucleic acid molecule that encodes a nucleotide sequence of SEQ ID NO: 5 or a part of it or a nucleotide having 90% sequence identity with said SEQ ID NO:5;
f) a nucleic acid molecule that encodes a nucleotide sequence of SEQ ID NO: 6 or a part of it or a nucleotide having 90% sequence identity with said SEQ ID NO:6; and
g) an internal control gene.

12. The test kit as claimed in claim 11, wherein said nucleotide sequence enables detection of Hepatitis 13 virus present in low copy number up to 60 IU/ml.

13. The test kit as claimed in claim 11, wherein said nucleotide sequence enables detection of Hepatitis B virus present in low copy number up to 20 IU/ml.

14. The test kit as claimed in claim 11, further comprising a reaction mixture comprising:
a) Nuclease free Water present in an amount of 0.95 μl;
b) Reverse transcript buffer present in an amount of 6.25 μl;
c) HCV Primer Forward present in an amount of 0.35 μl;
d) HCV Primer Reverse present in an amount of 0.35 μl;
e) HBV Primer Forward present in an amount of 0.35 μl;
f) HBV Primer Reverse present in an amount of 0.35 μl;
g) β-actin Primer Forward present in an amount of 0.20 μl;
h) β-actin Primer Reverse present in an amount of 0.20 μl;
i) HCV Probe present in an amount of 0.175 μl;
j) HBV Probe present in an amount of 0.175 μl;
k) fl-actin Probe present in an amount of 0.15 μl;
l) Reverse Transcriptase present in an amount of 0.5 μl; and
m) Template present in an amount of 7.50 μl.

15. The test kit as claimed in claim 11, wherein the HCV Probe comprises FAM and IABkFQ.

16. The test kit as claimed in claim 11, wherein the HBV Probe comprises Cy5 and IAbRQSp.

17. The test kit as claimed in claim 11, wherein the β-actin Probe comprises HEX and IABkFQ.

18. The test kit as claimed in claim 11, wherein said hepatitis virus is selected from the group comprising of hepatitis B virus, hepatitis C virus or combination thereof.

19. The test kit as claimed in claim 11, wherein SEQ ID No. 1 and 2 respectively represents forward and reverse primer to amplify hepatitis B virus.

20. The test kit as claimed in claim 11, wherein SEQ ID No. 3 represents probes for the detection of hepatitis 13 virus.

21. The test kit as claimed in claim 11, wherein SEQ ID No. 4 and 5 respectively represents forward and reverse primer to amplify hepatitis C virus.

22. The test kit as claimed in claim 11, wherein SEQ ID No. 6 represents probes for the detection of hepatitis C virus.

23. The test kit as claimed in claim 11, wherein said internal control comprises β actin gene.

24. The test kit as claimed in claim 11, wherein said probes and primers for Hepatitis B virus correspond to the S gene.

25. The test kit as claimed in claim 11, wherein said probes and primers for Hepatitis C virus correspond to the 5′ non-translated region.

26. The test kit as claimed in claim 11, sensitive to detect a sample with the viral load of 20 IU/ml.

27. The test kit as claimed in claim 11 wherein said nucleotide sequences enable high detection of all genotypes of Hepatitis B and C virus.
